(19) United States
(12) Patent Application Publication
(10) Pub. No.: US 2009/0036784 A1
(43) Pub. Date: Feb. 5, 2009

(54) DETERMINATION OF HEMODYNAMIC PARAMETERS

(75) Inventor: Ting Y. Lee, London (CA)

Correspondence Address: SMART AND BIGGAR, 438 UNIVERSITY AVENUE, SUITE 1500 BOX 111, TORONTO, ON, M5G2K8, CA

(73) Assignee: ROBARTS RESEARCH INSTITUTE

(21) Appl. No.: 11/573,992

(22) PCT Filed: Aug. 22, 2005
(86) PCT No.: PCT/CA2005/001305

§ 371 Date: Feb. 20, 2007

Related U.S. Application Data
(60) Provisional application No. 60/603,265, filed on Aug. 23, 2004.

(51) Int. Cl. A61B 5/02

(52) U.S. Cl. 600/481

(57) ABSTRACT

Hemodynamic parameters of an organ are determined by first estimating hemodynamic parameters for portions of an organ from time sequenced images of the portions obtained after injection of a contrast agent. For each of the portions, the accuracy of the estimated hemodynamic parameters is assessed based on at least one of (i) a relationship between extraction efficiency product (FE) and contrast distribution volume in interstitial space (Vₑ); (ii) a relationship between blood plasma space volume (Vₚ), FE, and Vₑ; and (iii) a value of contrast distribution volume (V_{D}).

### BACKGROUND

This invention relates to the determination of hemodynamic parameters.

Blood flow through a healthy organ may change in the event of a compromising event. The nature of the change of hemodynamic parameters can indicate the viability of the affected organ and, hence, the indicated intervention. For example, a coronary obstruction may impact myocardial hemodynamic parameters.

### SUMMARY OF INVENTION

According to the present invention, there is provided a method of determining hemodynamic parameters of an organ, comprising estimating hemodynamic parameters for portions of an organ from time sequenced images of the portions obtained after injection of a contrast agent. For each of the portions, the accuracy of the estimated hemodynamic parameters is assessed based on at least one of (i) a relationship between extraction efficiency product (FE) and contrast distribution volume in interstitial space (Vₑ); (ii) a relationship between blood plasma space volume (Vₚ), FE, and Vₑ; and (iii) a value of contrast distribution volume (V_{D}).

The hemodynamic parameters may be iteratively estimated where each estimate assumes the hemodynamic parameters are positively valued.

The estimated hemodynamic parameters may be determined, in part, from tissue contrast enhancement measured from the images. After obtaining estimated hemodynamic parameters, tissue contrast enhancement may be estimated based and these hemodynamic parameters. Any difference between the measured tissue contrast enhancement and the estimated tissue contrast enhancement may be considered an error factor which may be used as a correction factor for the estimated hemodynamic parameters.

Other features and advantages of the invention will become apparent from the following detailed description in conjunction with the drawings.

### BRIEF DESCRIPTION OF DRAWINGS

In the figures which illustrate an example embodiment of this invention,

FIG. 1 illustrates a compartmental model of an organ,

FIG. 2 is a graph of tissue contrast enhancement versus time, and

FIG. 3 is a graph of aortic contrast enhancement versus time.

### DETAILED DESCRIPTION

Blood flows through a living organ. Therefore, the present invention begins with the expectation that hemodynamic parameters of an organ (or portions thereof) may be determined by introducing a contrast agent into the organ and thereafter obtaining a plurality of time sequences images of the organ (or portions thereof).

A model of the organ is assumed in which (intravenously) injected contrast agent distributes itself within the organ in two compartments, namely, the blood space and the interstitial space (plus the intracellular space of the cells if their cellular membranes become injured due to ischemia). The blood space can be further simplified to the blood plasma space because contrast is generally excluded from entry into blood cells.

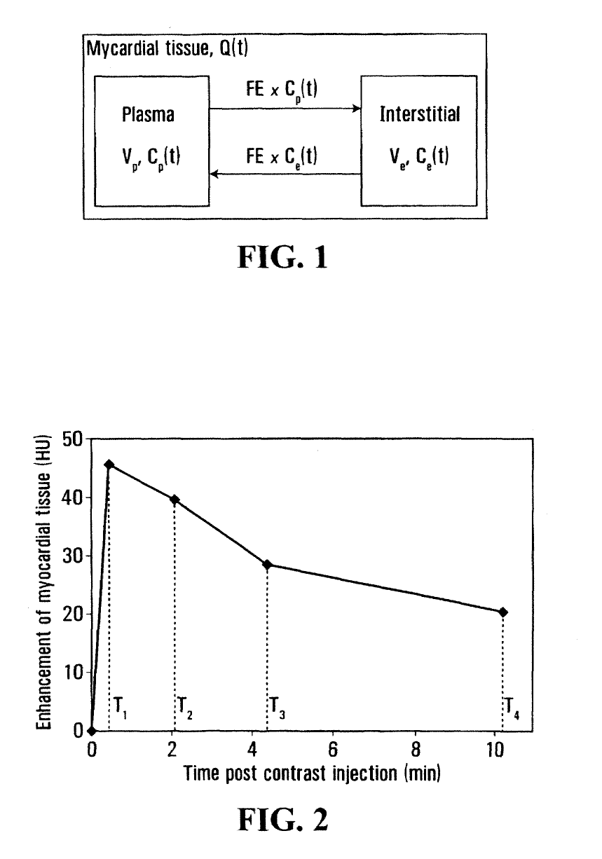

Given that the organ is the heart, any mass of myocardial tissue may be represented as shown in FIG. 1.

The symbols in the above model (FIG. 1) of the myocardium with respect to contrast distribution following intravenous administration are defined as follows:

FE the blood flow and extraction efficiency product governs the rate of transport of contrast between the blood plasma and interstitial space. It has units of blood flow or ml·min⁻¹·g⁻¹ and can be interpreted as FE ml of either blood plasma or interstitial fluid per min per gram of myocardial tissue that will be completely cleared of contrast. For compartmental models, blood flow (F) and extraction efficiency (E) are always tightly coupled as a product and each cannot be determined separately from the other. This is a major drawback of compartmental models. However, FE may still be useful as an estimate or a surrogate for blood flow provided the limitations are clearly understood: (1) it is less than blood flow depending on the value of extraction efficiency, which is always less than one; (2) in normal myocardium, extraction efficiency may be homeogeneous, however, this may not be the case in heart attack, where ischemic myocardium may have different E from normal myocardium and within ischemic myocardium, E may be quite heterogeneous.

E Extraction efficiency is the fraction of contrast present in blood plasma at arterial inlets to the myocardium that leaks into the interstitial space by the time blood plasma leaves from venous outlets of the myocardium. Extraction efficiency, blood flow (F) and capillary permeability surface product (PS) are related via the following relationship:

$$E = 1 - {e}^{- \frac{PS}{F}} or , PS = - F \cdot ln ( 1 - E )$$

Q(t) In the context of contrast enhanced computer tomography (CT), Q(t) is the enhancement expressed in Housfield units (HU) of the myocardial tissue at time t following contrast injection. In our model, the tissue enhancement Q(t) is, of course, make up of two parts. First, enhancement in the blood space, which is the product of the blood plasma space volume (Vₚ) and the blood plasma enhancement at time t (Cₚ(t)). Second, enhancement in the interstitial space, which is the product of the interstitial space volume (Vₑ, more strictly it should be contrast distribution volume in the interstitial space) and the enhancement of the interstitial space at time t (Cₑ(t)):

Q(t)=VₚCₚ(t)+VₑCₑ(t)
Note that contrast enhanced CT measures Q(t) in the myocardium and Cₚ(t) in blood vessels.

V_{D} It is the contrast distribution volume in the myocardium. This volume is the sum of Vₚ and Vₑ, i.e. V_{D}=Vₚ+Vₑ. Vₚ is the blood plasma volume in the myocardium. For normal myocardium, Vₑ is the distribution volume of contrast in the interstitial space. For abnormal myocardium, besides the distribution volume in the interstitial space, Vₑ also includes the distribution space within the myocytes when their cell membrane becomes permeable to contrast.

In one embodiment of the present invention, the goal is to determine the hemodynamic parameters FE, Vₚ and V_{D} using time lapsed sequences of coronary CT angiography.

The utilities of hemodynamic parameters are summarized as follows:

FE is a surrogate measure of myocardial perfusion. In acute or chronic MI, it indicates the severity of a coronary obstruction as well as the presence or absence of collateral circulation to the territory of the stenosed or occluded coronary. In the follow-up of reperfusion intervention, it can document whether the intervention is successful or not.

Vₚ The physiological mechanism of autoregulation would dictate that with decrease in myocardial perfusion, a viable myocardium would vasodilate to compensate for the decrease in perfusion, leading to an unchanged or elevated Vₚ. Conversely, a non-viable ischemic myocardium would have lost this autoregulatory ability such that Vₚ would start to decline from normal values. In other words, we can use the following mismatch matrix to differentiate between viable and non-viable ischemic myocardium

| | Ischemic viable | Ischemic non-viable |
|---|---|---|
| FE | − | −− |
| Vₚ | Unchanged/+ | −− |

− decreased from normal
−− large decrease from normal
+ increased from normal

V_{D} Normal myocardium has a V_{D} of 0.3-0.4 ml·g⁻¹. Injured myocardium (i.e. cell membrane of myocytes becomes permeable to contrast) has a higher V_{D} than normal. As injured myocardium recovers or remodels, V_{D} would return to normal levels.

In order to image a whole organ with CT, a series of images may be acquired, with each image representing a thin slice through the organ. The “slices” are parallel to each other and spaced from one another so that the series of images, taken together, represent the whole organ. Each image slice has a thickness (of about 5 mm). Each image slice is represented by a matrix of pixel values, with each pixel representing a volume of about 2 ml square and 5 ml thick. Thus, each pixel, because it represents a volume, may be referred to as a voxel.

In one approach according to this invention, the organ is scanned four separate times to acquire four series of images covering the organ of interest. These four times may be at 25 s (T₁), 1.5 min (T₂), 4 min (T₃), and 10 min (T₄) following contrast injection. (Actually, these four times are average times since it takes a short period of time to complete each CT scan in order to acquire one full series of images.) Myocardial tissue enhancement (Q(t)) may be measured for each voxel of each image at these four time points. Thus, for example, FIG. 2 shows tissue contrast enhancement measured for one voxel in a given image slice at four time points after contrast injection. FIG. 2 resulted from injection of 40 ml of contrast into a 29 kg dog at 2 ml/s and using a scanning protocol described hereafter.

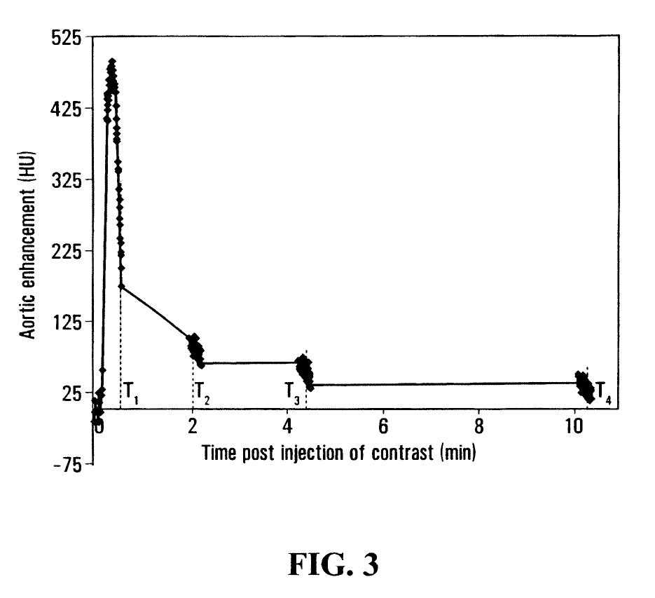

In order to measure arterial (aortic) enhancement (Cₚ(t)), the organ can be scanned continuously, or at short time intervals, for a brief time shortly after contrast injection in order to capture the expected contrast peak. In this regard if, as is conventional, the images are transverse images so that each image “cuts” through the aorta, aortic contrast enhancement could be determined using any single image plane, since the aortic contrast enhancement should be relatively invariant along the length of the aorta. After the peak, the aortic enhancement curve decreases exponentially and is very well characterised by the subsequent time points at 1.5, 4, and 10 min post injection. In this regard, since each image slice in a single scan may be considered to represent an image showing the same aortic enhancement, each of the image slices from a given scan at a time point may be used in establishing the aortic enhancement for that time point. FIG. 3 shows aortic contrast enhancement measured for one aortic voxel, with an initial continuous scan followed by measurements at three time points after contrast injection (using the series of images acquired for determination of tissue contrast enhancement). FIG. 3 also resulted from injection of 40 ml of contrast into a 29 kg dog at 2 ml/s and using the scanning protocol described hereafter.

The Mass balance for interstitial space leads to:

$${V}_{e} \frac{d {C}_{e} ( t )}{d t} = {FEC}_{p} ( t ) - {FEC}_{e} ( t )$$

$$\frac{d {C}_{e} ( t )}{d t} + \frac{FE}{{V}_{e}} {C}_{e} ( t ) = \frac{FE}{{V}_{e}} {C}_{p} ( t )$$

$${C}_{e} ( t ) = \frac{FE}{{V}_{e}} {C}_{p} ( t ) * {e}^{- \frac{FE}{{V}_{e}} t}$$

$$Q ( t ) = {V}_{e} {C}_{e} ( t ) + {V}_{p} {C}_{p} ( t ) \\ Q ( t ) = {FEC}_{p} ( t ) * {e}^{- \frac{FE}{{V}_{e}} t} + {V}_{p} {C}_{p} ( t ) = {FEC}_{p} ( t ) * {e}^{- kt} + {V}_{p} {C}_{p} ( t ) \\ k = \frac{FE}{{V}_{e}} \\ \begin{matrix}{\int }_{0}^{T} Q ( t ) d t = FE \cdot {\int }_{0}^{T} {C}_{p} ( t ) * {e}^{- kt} d t + {V}_{p} {\int }_{0}^{T} {C}_{p} ( t ) d t \\ = FE \cdot {\int }_{0}^{T} d t {\int }_{0}^{t} {C}_{p} ( u ) {e}^{- k ( t - u )} d u + {V}_{p} {\int }_{0}^{T} {C}_{p} ( t ) d t \\ = FE \cdot {\int }_{0}^{T} d u {\int }_{u}^{T} d {tC}_{p} ( u ) {e}^{- k ( t - u )} + {V}_{p} {\int }_{0}^{T} {C}_{p} ( t ) d t \\ = FE \cdot {\int }_{0}^{T} {C}_{p} ( u ) d u {{\int }_{0}^{T} {e}^{- k ( t - u )} d}_{{1 \; 4 \; 2 \; 43}_{t = t - u}} t + {V}_{p} {\int }_{0}^{T} {C}_{p} ( t ) d t \\ = FE \cdot {\int }_{0}^{T} {C}_{p} ( u ) d u {\int }_{0}^{T - u} {e}^{- {kt}^{′}} d {t}^{′} + {V}_{p} {\int }_{0}^{T} {C}_{p} ( t ) d t \\ = FE \cdot {\int }_{0}^{T} {C}_{p} ( u ) d u ( - {\frac{1}{k} [ {e}^{- {kt}^{′}} ]}_{0}^{T - u} ) + {V}_{p} {\int }_{0}^{T} {C}_{p} ( t ) d t \\ = \frac{FE}{k} ( 1 - {e}^{- k ( t - u )} ) {\int }_{0}^{T} {C}_{p} ( u ) d u + {V}_{p} {\int }_{0}^{T} {C}_{p} ( t ) d t \\ = \frac{FE}{k} {\int }_{0}^{T} {C}_{p} ( u ) d u - \frac{FE}{k} {\int }_{0}^{T} {C}_{p} ( u ) {e}^{- k ( T - u )} d u + \\ {V}_{p} {\int }_{0}^{T} {C}_{p} ( t ) d t \\ = \frac{FE}{k} {\int }_{0}^{T} {C}_{p} ( t ) d t - {\frac{FE}{k} [ {C}_{p} ( t ) * {e}^{- kt} ]}_{t = T} + \\ {V}_{p} {\int }_{0}^{T} {C}_{p} ( t ) d t \\ = \frac{FE}{k} {\int }_{0}^{T} {C}_{p} ( t ) d t - \frac{1}{k} ( Q ( T ) - {V}_{p} {C}_{p} ( T ) ) + \\ {V}_{p} {\int }_{0}^{T} {C}_{p} ( t ) d t\end{matrix}$$

### Therefore,

$${\int }_{0}^{T} Q ( t ) d t = ( \frac{FE}{k} + {V}_{p} ) {\int }_{0}^{T} {C}_{p} ( t ) d t - \frac{1}{k} Q ( T ) + \frac{{V}_{p}}{k} {C}_{p} ( T )$$

$$Let , \\ {A}_{Q} ( T ) = {\int }_{0}^{T} Q ( t ) d t$$

$${A}_{p} ( T ) = {\int }_{0}^{T} {C}_{p} ( t ) d t ,$$

In other words, A_{Q}(T) and Aₚ(T) are the areas underneath the tissue and aortic enhancement vs time curves to time T. Then:

$${A}_{Q} ( T ) = ( \frac{FE}{k} + {V}_{p} ) {A}_{p} ( T ) - \frac{1}{k} Q ( T ) + \frac{{V}_{p}}{k} {C}_{p} ( T ) \qquad ( 1 )$$

Suppose we have measurements of Q(T) at T₁, T₂, T₃ and T₄ and measurement of Cₚ(t) at higher temporal frequency, then

$${A}_{Q} ( {T}_{1} ) = ( \frac{FE}{k} + {V}_{p} ) {A}_{p} ( {T}_{1} ) - \frac{1}{k} Q ( {T}_{1} ) + \frac{{V}_{b}}{k} {C}_{p} ( {T}_{1} )$$

$${A}_{Q} ( {T}_{2} ) = ( \frac{FE}{k} + {V}_{p} ) {A}_{p} ( {T}_{2} ) - \frac{1}{k} Q ( {T}_{2} ) + \frac{{V}_{b}}{k} {C}_{p} ( {T}_{2} )$$

$${A}_{Q} ( {T}_{3} ) = ( \frac{FE}{k} + {V}_{p} ) {A}_{p} ( {T}_{3} ) - \frac{1}{k} Q ( {T}_{3} ) + \frac{{V}_{b}}{k} {C}_{p} ( {T}_{3} )$$

$${A}_{Q} ( {T}_{4} ) = ( \frac{FE}{k} + {V}_{p} ) {A}_{p} ( {T}_{4} ) - \frac{1}{k} Q ( {T}_{4} ) + \frac{{V}_{b}}{k} {C}_{p} ( {T}_{4} )$$

in matrix form:

$$[ \begin{matrix}{A}_{Q} ( {T}_{1} ) \\ {A}_{Q} ( {T}_{2} ) \\ {A}_{Q} ( {T}_{3} ) \\ {A}_{Q} ( {T}_{4} )\end{matrix} ] = [ \begin{matrix}{A}_{p} ( {T}_{1} ) & - Q ( {T}_{1} ) & {C}_{p} ( {T}_{1} ) \\ {A}_{p} ( {T}_{2} ) & - Q ( {T}_{2} ) & {C}_{p} ( {T}_{2} ) \\ {A}_{p} ( {T}_{3} ) & - Q ( {T}_{3} ) & {C}_{p} ( {T}_{3} ) \\ {A}_{p} ( {T}_{4} ) & - Q ( {T}_{4} ) & {C}_{p} ( {T}_{4} )\end{matrix} ] [ \begin{matrix}\frac{FE}{k} + {V}_{p} \\ {k}^{- 1} \\ {V}_{p} \cdot {k}^{- 1}\end{matrix} ] \qquad ( 2 )$$

### Since:

$$\frac{FE}{k} = {V}_{e} and \frac{FE}{k} + {V}_{p} = {V}_{e} + {V}_{p} = {V}_{D} , \qquad Eq ( 1 )$$

can be rewritten as:

$$[ \begin{matrix}{A}_{Q} ( {T}_{1} ) \\ {A}_{Q} ( {T}_{2} ) \\ {A}_{Q} ( {T}_{3} ) \\ {A}_{Q} ( {T}_{4} )\end{matrix} ] = [ \begin{matrix}{A}_{p} ( {T}_{1} ) & - Q ( {T}_{1} ) & {C}_{p} ( {T}_{1} ) \\ {A}_{p} ( {T}_{2} ) & - Q ( {T}_{2} ) & {C}_{p} ( {T}_{2} ) \\ {A}_{p} ( {T}_{3} ) & - Q ( {T}_{3} ) & {C}_{p} ( {T}_{3} ) \\ {A}_{p} ( {T}_{4} ) & - Q ( {T}_{4} ) & {C}_{p} ( {T}_{4} )\end{matrix} ] [ \begin{matrix}{V}_{D} \\ {k}^{- 1} \\ {V}_{p} \cdot {k}^{- 1}\end{matrix} ] \qquad ( 3 )$$

where V_{D} is the contrast distribution volume in the myocardium. Eq (3) can be solved using non-negative least squares (NNLS) for the three parameters V_{D}, k⁻¹ and Vₚ·k⁻¹. Since it is physiologically not possible for V_{D}, k⁻¹ and Vₚ·k⁻¹ to become negative, the NNLS algorithm has the advantage over the traditional linear linear squares method in that the estimated parameters are constrained to be larger than or equal to zero. From these estimates, the desired parameters: V_{D}, Vₚ and FE can be derived as:

V_{D} already estimated from the NNLS solution of Eq (3)

$$\begin{matrix}{V}_{p} = \frac{{V}_{p} \cdot {k}^{- 1}}{{k}^{- 1}} & ( 3 A ) \\ FE = \frac{{V}_{D} - {V}_{p}}{{k}^{- 1}} & ( 3 B )\end{matrix}$$

Note that the above system of linear equations for the parameters V_{D}, k⁻¹ and Vₚ·k⁻¹ is derived without the assumption that the “backflux” of contrast from the interstitial space to the blood space is negligible (i.e. without the assumption of the Patlak graphical analysis).

There is (additive) measurement noise in Q(t) such that the expression for Q(t) should be written as:

Q(t)=FECₚ(t)*e^{-kt}+VₚCₚ(t)+ε(t)

After lineariziation, the equation becomes:

$${A}_{Q} ( T ) = ( \frac{FE}{k} + {V}_{p} ) {A}_{p} ( T ) - \frac{1}{k} Q ( T ) + \frac{{V}_{p}}{k} {C}_{p} ( T ) + \frac{ɛ ( T )}{k} + {\int }_{0}^{T} ɛ ( t ) d t$$

where ε(t) is a zero mean Gaussian process.
Eq (2) then becomes:

$$[ \begin{matrix}{A}_{Q} ( {T}_{1} ) \\ {A}_{Q} ( {T}_{2} ) \\ {A}_{Q} ( {T}_{3} ) \\ {A}_{Q} ( {T}_{4} )\end{matrix} ] = [ \begin{matrix}{A}_{p} ( {T}_{1} ) & - Q ( {T}_{1} ) & {C}_{p} ( {T}_{1} ) \\ {A}_{p} ( {T}_{2} ) & - Q ( {T}_{2} ) & {C}_{p} ( {T}_{2} ) \\ {A}_{p} ( {T}_{3} ) & - Q ( {T}_{3} ) & {C}_{p} ( {T}_{3} ) \\ {A}_{p} ( {T}_{4} ) & - Q ( {T}_{4} ) & {C}_{p} ( {T}_{4} )\end{matrix} ] [ \begin{matrix}{V}_{D} \\ {k}^{- 1} \\ {V}_{p} \cdot {k}^{- 1}\end{matrix} ] + \frac{1}{k} [ \begin{matrix}ɛ ( {T}_{1} ) \\ ɛ ( {T}_{2} ) \\ ɛ ( {T}_{3} ) \\ ɛ ( {T}_{4} )\end{matrix} ] + [ \begin{matrix}{A}_{ɛ} ( {T}_{1} ) \\ {A}_{ɛ} ( {T}_{2} ) \\ {A}_{ɛ} ( {T}_{3} ) \\ {A}_{ɛ} ( {T}_{4} )\end{matrix} ] \qquad ( 4 )$$

where

$${A}_{ɛ} ( {T}_{1} ) = {\int }_{0}^{{T}_{1}} ɛ ( t ) d t$$

$${A}_{ɛ} ( {T}_{2} ) = {\int }_{0}^{{T}_{2}} ɛ ( t ) d t$$

$${A}_{ɛ} ( {T}_{3} ) = {\int }_{0}^{{T}_{3}} ɛ ( t ) d t$$

$${A}_{ɛ} ( {T}_{4} ) = {\int }_{0}^{{T}_{4}} ɛ ( t ) d t$$

Since ε(t) is a zero mean Gaussian process, each A_{ε}(T) is also a zero mean Gaussian process. Except for the error vector

$${\overline{E}}_{R} = \frac{1}{k} [ \begin{matrix}ɛ ( {T}_{1} ) \\ ɛ ( {T}_{2} ) \\ ɛ ( {T}_{3} ) \\ ɛ ( {T}_{4} )\end{matrix} ] \qquad ( 5 )$$

Eq (5) is the formulation of a least squares problem for the estimation of V_{D}, k⁻¹, Vₚ·k⁻¹. To account for the error vector, an iterative least squares procedure can be used. The algorithm is as follows:

1. estimate V_{D}, k⁻¹, Vₚ·k⁻¹ with the error vector, Ē_{R}, set to zero.

2. from the estimated (V_{D}, k⁻¹, Vₚ·k⁻¹), (FE, Vₚ, k) are calculated and used to estimate Q(T₁), Q(T₂), Q(T₃), and Q(T₄) from the following equation:

Q(t)=FECₚ(t)*e^{-kt}+VₚCₚ(t)
The difference of the estimated and measured Q(Tᵢ) gives ε(Tᵢ), i=1,2,3,4.

3. The error vector, Ē_{R}, is calculated from Eq (5) and subtracted from the right hand side of Eq (4).

4. A new set of (V_{D}, k⁻¹, Vₚ·k⁻¹) is estimated and steps 1-4 are repeated until convergence.

There are three special cases to consider:

Case (1): k⁻¹ is small, or k tends to infinity, then Vₑ<<FE. This means there is very little leakage of contrast, such as where the voxel is in a blood vessel.
Eq (3), in the limit of k is large, reduces to:

$$[ \begin{matrix}{A}_{Q} ( {T}_{1} ) \\ {A}_{Q} ( {T}_{2} ) \\ {A}_{Q} ( {T}_{3} ) \\ {A}_{Q} ( {T}_{4} )\end{matrix} ] = {V}_{D} [ \begin{matrix}{A}_{p} ( {T}_{1} ) \\ {A}_{p} ( {T}_{2} ) \\ {A}_{p} ( {T}_{3} ) \\ {A}_{p} ( {T}_{4} )\end{matrix} ] \qquad ( 6 )$$

Since the algorithm is to estimate V_{D}, k⁻¹, Vₚ·k⁻¹ from Eq (3), this means that when k⁻¹ is zero, the sensitivity for Vₚ·k⁻¹ is very small, so it should be ignored. The estimate of V_{D} should be set to Vₚ because:

$$k {e}^{- k t} {\to }^{k \to ∞} δ ( t )$$

$$and , \\ \begin{matrix}Q ( t ) = {FEC}_{p} ( t ) * {e}^{- kt} + {V}_{p} {C}_{p} ( t ) \\ = \frac{FE}{k} {C}_{p} ( t ) * k \cdot {e}^{- kt} + {V}_{p} {C}_{p} ( t ) {\to }^{k \to ∞} {V}_{e} {C}_{p} ( t ) * δ ( t ) + {V}_{p} {C}_{p} ( t ) \\ = ( {V}_{e} + {V}_{p} ) \cdot {C}_{p} ( t ) \\ \approx {V}_{p} {C}_{p} ( t )\end{matrix}$$

Therefore, if k is large, there is no leakage of contrast into the interstitial space and Q(t) is just the product of Vₚ and Cₚ(t) or

A_{Q}(t)=Vₚ·Aₚ(t)

implying that in Eq (6), V_{D} is actually Vₚ.
Case (2): k⁻¹ is large, or k tends to zero (indicating blow flow is low, such as in scar tissue), then:

$$Q ( t ) = {FEC}_{p} ( t ) * {e}^{- kt} + {V}_{p} {C}_{p} ( t ) {\to }^{k \to 0} FE {\int }_{0}^{t} {C}_{a} ( s ) d s + {V}_{p} {C}_{p} ( t )$$

This is the Patlak and Blasberg model for the case when there is no back flux of contrast from the interstitial space to the plasma space.

$$\begin{matrix} {\int }_{0}^{T} Q ( t ) d t = FE \cdot {\int }_{0}^{T} {C}_{p} ( t ) * {e}^{- kt} d t + {V}_{p} {\int }_{0}^{T} {C}_{p} ( t ) d t \\ {\to }^{k \to 0} FE \cdot {\int }_{0}^{T} d t {\int }_{0}^{t} {C}_{p} ( u ) d u + {V}_{p} {\int }_{0}^{T} {C}_{p} ( t ) d t \\ = FE \cdot {\int }_{0}^{T} d u {\int }_{u}^{T} d {tC}_{p} ( u ) + {V}_{p} {\int }_{0}^{T} {C}_{p} ( t ) d t \\ = FE \cdot {\int }_{0}^{T} {C}_{p} ( u ) d u {\int }_{u}^{T} d t + {V}_{p} {\int }_{0}^{T} {C}_{p} ( t ) d t \\ = FE \cdot {\int }_{0}^{T} {C}_{p} ( u ) d u ( T - u ) + {V}_{p} {\int }_{0}^{T} {C}_{p} ( t ) d t\end{matrix} \\ or , \\ {A}_{q} ( T ) = FE \cdot T \cdot {\int }_{0}^{T} {C}_{p} ( u ) d u - FE \cdot {\int }_{0}^{T} u \cdot {C}_{p} ( u ) d u + {V}_{p} {\int }_{0}^{T} {C}_{p} ( u ) d u \qquad ( 7 )$$

It can be shown, in the case k tends to zero, that Eq (1) reduces to Eq (7):

$$\begin{matrix}{A}_{Q} ( T ) = ( \frac{FE}{k} + {V}_{p} ) {A}_{p} ( T ) - \frac{1}{k} Q ( T ) + \frac{{V}_{p}}{k} {C}_{p} ( T ) \\ = \frac{FE}{k} {A}_{p} ( T ) + {V}_{p} {A}_{p} ( T ) - \frac{1}{k} Q ( T ) + \frac{{V}_{p}}{k} {C}_{p} ( T ) \\ = \frac{FE}{k} {A}_{p} ( T ) + {V}_{p} {A}_{p} ( T ) - \frac{1}{k} ( Q ( T ) - {V}_{p} {C}_{p} ( T ) ) \\ = \frac{FE}{k} {A}_{p} ( T ) + {V}_{p} {A}_{p} ( T ) - {\frac{FE}{k} [ {C}_{p} ( t ) * {e}^{- kt} ]}_{t = T} \\ = \frac{FE}{k} {\int }_{0}^{T} {C}_{p} ( u ) d u + {V}_{p} {A}_{p} ( T ) - \frac{FE}{k} {\int }_{0}^{T} {C}_{p} ( u ) {e}^{- k ( T - u )} d u \\ = \frac{FE}{k} {\int }_{0}^{T} [ 1 - {e}^{- k ( T - u )} ] \cdot {C}_{p} ( u ) d u + {V}_{p} \cdot {A}_{p} ( T )\end{matrix}$$

### Since

$${lim}_{k \to 0} \frac{1 - {e}^{- k ( T - u )}}{k} = T - u ,$$

$${A}_{Q} ( T ) = FE \cdot T \cdot {\int }_{0}^{T} {C}_{p} ( u ) d u - FE \cdot {\int }_{0}^{T} u \cdot {C}_{p} ( u ) d u + {V}_{p} {\int }_{0}^{T} {C}_{p} ( u ) d u \qquad ( 8 )$$

the same as Eq (1).
To verify that the algorithm still performs in this special case (k→0 ), a theoretical Q(t) with k=0 is constructed with a given Cₚ(t). The algorithm (Eq (3)) is used to solve for the set of parameters (V_{D}, k⁻¹, Vₚ·k⁻¹) and the estimated parameters are compared to their true values. It was found from simulation tests that when k→0, the solution of Eq (3) could lead to estimates of:

$$\frac{1}{k} \neq 0 but \frac{{V}_{p}}{k} = 0.$$

Thus, when Eq (3) produces the above estimates, Eq (8) should be used instead for A_{Q}(t):

$$Let {M}_{p} ( T ) = {\int }_{0}^{T} u \cdot {C}_{p} ( u ) d u$$

Then, the equations for A_{Q}(t) at t=T₁, T₂, T₃, T₄ can be written as the following matrix equation:

$$[ \begin{matrix}{A}_{Q} ( {T}_{1} ) \\ {A}_{Q} ( {T}_{2} ) \\ {A}_{Q} ( {T}_{3} ) \\ {A}_{Q} ( {T}_{4} )\end{matrix} ] = [ \begin{matrix}{T}_{1} \cdot {A}_{p} ( {T}_{1} ) - {M}_{p} ( {T}_{1} ) & {A}_{p} ( {T}_{1} ) \\ {T}_{2} \cdot {A}_{p} ( {T}_{2} ) - {M}_{p} ( {T}_{2} ) & {A}_{p} ( {T}_{2} ) \\ {T}_{3} \cdot {A}_{p} ( {T}_{3} ) - {M}_{p} ( {T}_{3} ) & {A}_{p} ( {T}_{3} ) \\ {T}_{4} \cdot {A}_{p} ( {T}_{4} ) - {M}_{p} ( {T}_{4} ) & {A}_{p} ( {T}_{4} )\end{matrix} ] \cdot [ \begin{matrix}FE \\ {V}_{p}\end{matrix} ] \qquad ( 9 )$$

Eq (9) can then be solved for FE and Vₚ as before with the NNLS algorithm.

### Case (3):

$$\frac{FE}{k} \approx 0 and {V}_{p} \approx 0 but \frac{FE}{k} << {V}_{p} ,$$

(which, like case (1), indicates there is very little leakage of contrast),

Q(t)→Vₚ·Cₚ(t), which is similar to case (1) above.

To investigate the behavior of Eq (3) in this special case, a number of theoretical Q(t)'s with parameters from the following table are constructed with a given Cₚ(t).

| FE (ml/min/g) | k (s) | $\frac{FE}{k} ( ml \text{/} g )$ | Vₚ(ml/g) |
|---|---|---|---|
| 0.2 | 0.3 | 0.011 | 0.05 |
| 0.2 | 0.4 | 0.0083 | 0.05 |
| 0.2 | 0.5 | 0.0067 | 0.05 |

Eq (3) is used to solve for the set of parameters (V_{D}, k⁻¹, Vₚ·k⁻¹) and the estimated parameters are compared to their true values. It was found from these simulation tests that the solution of Eq (3) lead to estimates of:

$${V}_{D} = 0$$

$${k}^{- 1} \neq 0$$

$$\frac{{V}_{p}}{k} \neq 0$$

The above tests suggest that when an estimate of V_{D}=0 is returned with Eq (3), Eq (6) should be used instead with V_{D} set to Vₚ as in case (1).

Interactions (Covariances) Among the Set of Parameters (V_{D}, k⁻¹, Vₚ·k⁻¹).

Since Q(t) is modeled as the sum of two terms:

Q(t)=FE[·Cₚ(t)*e^{-k·t}┘+Vₚ·Cₚ(t)

changes in the second term Vₚ·Cₚ(t) can be offset by opposite changes in the first term FE·[Cₚ(t)*e^{-k·t}] to maintain the same quality of fit to Q(t). This is manifested as opposite changes in the estimated parameters Vₚ and FE, that is, the estimated values of Vₚ and FE are negatively correlated. From simulations it is determined that the parameter V_{D}, unlike Vₚ and FE, is more accurately estimated from Eq (3) and is more free of covariations with either Vₚ or FE.

The following strategy is adopted to overcome the co-variations among the estimates of Vₚ and FE and possibly V_{D}:

a) Estimates of (V_{D}, k⁻¹, Vₚ·k⁻¹) or equivalently (V_{D}, k, Vₚ) are obtained from Eq (3).

b) Check for:

$$\begin{matrix}\frac{1}{k} = 0 & Case ( 1 ) \\ \frac{{V}_{p}}{k} = 0 & Case ( 2 ) \\ {V}_{D} = 0 & Case ( 3 )\end{matrix}$$

This is actually checked by determining if the left hand side of each equation is less than a threshold value (so as to be very close to zero).
If either one of Cases (1)-(3) is satisfied, solve for
   Case (1) & (3), V_{D}=Vₚ from Eq (6)
   Case (2), FE and Vₚ from Eq (9)
and the procedure terminates

c) If none of these three special cases exist, assume the estimate of V_{D} is correct, and that only estimates of k and Vₚ are in error. Perform another optimization of the fit to A_{Q}(t) with k and Vₚ as the only two adjustable parameters.
i) Vₚ is varied, while k is kept unchanged at the original estimated value from a) above, until a best fit to Q(t) is obtained. Note that since

$${V}_{D} = \frac{FE}{k} + {V}_{p}$$

changing Vₚ with V_{D} and k fixed at the original estimates from a) above, means FE is changed with Vₚ.
ii) k is varied, while keeping Vₚ unchanged at the value found in b.i) above, until a best fit to Q(t) is obtained. As in i) above, since

$${V}_{D} = \frac{FE}{k} + {V}_{p}$$

changing k with V_{D} fixed at the original estimate from a) above and Vₚ fixed at the new estimate from b.i) above, means FE is changed with k.
At this point, a new set of estimates for (k, Vₚ) is obtained.

d) Use ‘Golden search’ to determine a new value for V_{D} and repeat steps c) and d) until convergence of ‘Golden search’ for V_{D}.

Covariance Matrix of the Model, Eq (3)

The covariances of the estimated parameters (V_{D}, k⁻¹, Vₚ·k⁻¹) are given by the covariance matrix:

Cov(V_{D}, k⁻¹, Vₚ·k⁻¹)=σ²·{[M_{F}]^{T}·M_{F}}⁻¹

where σ² is the variance of the measurements A_{Q}(t) and M_{F} is the Fisher information (sensitivity) matrix defined as:

$${M}_{F} = [ \begin{matrix}{A}_{p} ( {T}_{1} ) & - Q ( {T}_{1} ) & {C}_{p} ( {T}_{1} ) \\ {A}_{p} ( {T}_{2} ) & - Q ( {T}_{2} ) & {C}_{p} ( {T}_{2} ) \\ {A}_{p} ( {T}_{3} ) & - Q ( {T}_{3} ) & {C}_{p} ( {T}_{3} ) \\ {A}_{p} ( {T}_{4} ) & - Q ( {T}_{4} ) & {C}_{p} ( {T}_{4} )\end{matrix} ]$$

The variances and covariances of the estimated parameters are large when columns of M_{F} are similar. For example: (a) for case (1) and case (3), the 2^{nd} and 3^{rd} column are proportional to each other; and (b) for case (2), the 1^{st} and 2^{nd} column are similar.

Having described an approach to estimate hemodynamic parameters, a suitable protocol to implement the approach is next described.

### 1. Scout

set limits to cover the entire thorax and upper abdomen

2. Localization helical scan

prescribe a helical scan with breath hold from the scout to cover from carina to beyond the dome of the liver

3. Timing bolus

from the localization helical scan, select the level at the ascending aorta. Set up a 25 s cine scan of the chosen level at 1 s interval, 120 kVₚ, 50 mA, 5 s prep delay, collimation 1×10 mm. Inject 20 ml of contrast at 4 ml/s and start cine scan at the same time. This is simply to determine when aortic contrast enhancement peaks so as to know when to start a cine scan after injection of the main dose of contrast.

From the acquired timing bolus cine scan determine the time of peak enhancement at the ascending aorta, for example, 20 s after start of injection of contrast.

4. Coronary CT angiography performed with ECG-gated helical scan at ‘baseline’ (before injection of the main dose of contrast)

a ECG gated helical scan: 1.25 mm slice thickness at 1.25 mm interval, pitch 0.3 (0.3 mm/rotation), 0.5 s per rotation, 120 kVp, 75 mA to cover from carina to dome of liver with breath hold at 75% of R-R interval.

5. A non-ECG gated cine scan to acquire the initial portion of the aortic enhancement curve followed by coronary CT angiography performed with ECG-gated helical scan

Set up:

(a) a 15 s cine scan at the level of the carina at 1 s interval, 120 kVₚ, 50 mA, 1 s per rotation, 2 s prep delay, collimation 4×1.25 mm;

(b) intergroup delay 3 s

(c) a ECG gated helical scan: 1.25 mm slice thickness at 1.25 mm interval, pitch 0.3 (0.3 mm/rotation), 0.5 s per rotation, 120 kVₚ, 300 mA to cover from carina to dome of liver with breath hold at 75% of R-R interval.

Inject 120 ml of contrast at 4 ml/s starting at the same time as the start of the cine scan in (a) above.

6. ECG-gated coronary CT angiography at 1.5 minute after injection of contrast in step 4

Use same technique as the ECG gated helical scan in step 4 except x-ray tube current is lowered from 300 to 75 mA

7. ECG-gated coronary CT angiography at 4.0 minute after injection of contrast in step 4

Use same technique as the ECG gated helical scan in step 4 except x-ray tube current is lowered from 300 to 75 mA

8. ECG-gated coronary CT angiography at 10.0 minute after injection of contrast in step 4

Use same technique as the ECG gated helical scan in step 4 except x-ray tube current is lowered from 300 to 75 mA

Effective Dose Equivalents (H_{E})

Only effective dose equivalents for the baseline, 25 s, 1.5 min, 4 min and 10 min coronary CT angiograms as well as the cine scan are considered.

| | |
|---|---|
| Baseline coronary CT angiogram | 2.27 mSv |
| 15 s Cine scan | 0.42 mSv |
| 25 s post coronary CT angiogram | 9.07 mSv |
| 1.5 min post coronary CT angiogram | 2.27 mSv |
| 4.0 min post coronary CT angiogram | 2.27 mSv |
| 10.0 min post coronary CT angiogram | 2.27 mSv |
| Total | 18.57 mSv |

In comparison, the effective dose equivalent for a routine contrast-enhanced CT chest study consisting of a baseline and a non-enhanced CT scan is 24.2 mSv and for a 10 mCi FDG PET scan for myocardial viability, it is 7.2 mSv. The normal background radiation gives an annual effective dose equivalent of 2 mSv.

Analysis Steps

1. The ECG-gated coronary CT angiograms at baseline, 1.5 min, 4 min and 10 min post injection of contrast are registered in 3-D with respect to that at 25 sec post.
2. The cine scan from step 5(a) as well as the registered coronary CT angiograms are used to generate the aortic enhancement curve, Cₚ(t):

(a) cine scan in step 5 provides the first 2-17 s of data

(b) coronary angiogram in step 5 provides data from 20-44 s

(c) coronary angiogram in step 6 provides data from 1.5-1.9 min

(d) coronary angiogram in step 7 provides data from 4.0-4.4 min

(e) coronary angiogram in step 8 provides data from 10.0-10.4 min

all the time periods above are referenced to the start of injection of contrast in step 5. A ROI placed in the aorta is used to generate the aortic enhancement curve. For the coronary angiograms, the aortic ROI may have to be adjusted at each level of the aorta. Missing data in the time interval between successive coronary angiograms are recovered by linear interpolation.

3. The baseline coronary angiogram is subtracted from the delayed angiograms after contrast injection to generate the tissue enhancement curve, Q(t), for each pixel in the myocardium. Both the baseline and delayed angiograms are reformatted into the short-axis format before subtraction
4. Aₚ(T) and Cₚ(T) are determined from the measured aortic enhancement curve Cₚ(t). A_{Q}(T) and Q(T) are determined for each pixel (voxel) from the corresponding pixel enhancement curve. FE, V_{D} and Vₑ for each pixel are determined via Eq (3), (3A), and (3B) to generate the corresponding functional maps of the whole heart in short-axis format.

Pixel Timing Information in the Registered and Reformatted Raw CTA Images

The five sets of CTA images have to be registered with each other and then reformatted in the short-axis view of the LV (Analysis steps 1 and 2). Since Eq (3) requires the ‘acquisition’ time of each individual pixel, the registration and reformatting steps produce two problems, first the acquisition time of each pixel has to be determined and second, unlike the raw CTA images, the acquisition time of each pixel in a registered and reformatted image is not uniform. A simple method to generate the ‘acquisition’ time of each pixel is to create a new set of images for each CTA, in which the value of all pixels is equal to the mid-scan time of the CTA image. The registration and reformatting operations in analysis steps 1 and 2 are applied to in the same way to both the CTA images and the acquisition time images. As a result, the value of a pixel in the registered and reformatted acquisition time images will be the correct acquisition time of the pixel.

While the described technique used four measurement times, the technique requires only three measurement times since Eq (3) has three unknowns. However, there is a significant sacrifice of accuracy and precision if the estimation only uses three measurement times. While accuracy and precision will improve with the number of measurement times employed, the radiation dose to the patient increases with each scan. Therefore, it has been found that four measurement times (following a cine scan to capture peak aortic enhancement) might provide a reasonable compromise between accuracy and precision and radiation dose. However, depending on the circumstances, five, six, or more measurement times might be indicated.

While the described approach to obtain hemodynamic parameters of an organ have been described assuming that the organ is the heart, it will be apparent that the approach has equal application to other organs. Thus, for example, the approach may be used in obtaining hemodynamic parameters for the brain. In such instance, the scans may be from the top to the bottom of the brain and many of the resulting image slices will show the internal carotid artery or middle cerebral arteries so that blood contrast enhancement can be determined in the same manner as described for the determination of aortic contrast enhancement.

Also, while the described approach has been described in conjunction with CT scanning, equally any other suitable scanning technique may be used, such as magnetic resonance scanning.

Other modifications will be apparent to those skilled in the art and, therefore, the invention is defined in the claims.

1. A method of determining hemodynamic parameters of an organ, comprising:
estimating hemodynamic parameters for portions of an organ from time sequenced images of said portions obtained after injection of a contrast agent;
for each of said portions, assessing accuracy of said estimated hemodynamic parameters based on at least one of (i) a relationship between extraction efficiency product (FE) and contrast distribution volume in interstitial space (Vₑ); (ii) a relationship between blood plasma space volume (Vₚ), FE, and Vₑ; and (iii) a value of contrast distribution volume (V_{D}).

2. The method of claim 1 wherein said estimated hemodynamic parameters are considered inaccurate if said relationship between extraction efficiency product (FE) and contrast distribution volume in interstitial space (Vₑ) is such that the quotient of Vₑ/FE is within a threshold of zero.

3. The method of claim 1 wherein said estimated hemodynamic parameters are considered inaccurate if said relationship between blood plasma space volume (Vₚ), FE, and Vₑ is such that Vₚ·Vₑ/FE is within a threshold of zero.

4. The method of claim 1 wherein said estimated hemodynamic parameters are considered inaccurate if said value of contrast distribution volume (V_{D}) is such that V_{D} is within a threshold of zero.

5. The method of claim 2 wherein if said estimated hemodynamic parameters are considered inaccurate, re-estimating said hemodynamic parameters assuming Vₑ/FE has a value of zero.

6. The method of claim 3 wherein if said estimated hemodynamic parameters are considered inaccurate, re-estimating said hemodynamic parameters assuming Vₚ·Vₑ/FE has a value of zero.

7. The method of claim 4 wherein if said estimated hemodynamic parameters are considered inaccurate, re-estimating said hemodynamic parameters assuming V_{D} has a value of zero.

8. The method of claim 1 wherein said estimating comprises:
for each portion of each image, determining at least one of tissue contrast enhancement and blood plasma contrast enhancement;
for each image acquisition time, obtaining a measure related to an integral of tissue contrast enhancement with respect to time over a range ending with said each image acquisition time and obtaining a measure related to an integral of blood plasma contrast enhancement with respect to time over a range ending with said each image acquisition time.

9. The method of claim 8 wherein said estimating further comprises iteratively estimating said hemodynamic parameters, where each estimate assumes said hemodynamic parameters are positively valued.

10. The method of claim 8 further comprising, based on said estimated hemodynamic parameters, estimating an estimated tissue contrast enhancement, setting an error to a difference between determined tissue contrast enhancement determined from said determining and said estimated tissue contrast enhancement, and using said error as a correction factor for said estimated hemodynamic parameters.

11. The method of claim 8 further comprising from a baseline image of said organ free of said contrast agent, measuring a baseline tissue contrast and baseline blood plasma contrast for each of said portions of said organ.

12. The method of claim 11 wherein said determining at least one of tissue contrast enhancement and blood plasma contrast enhancement comprises measuring at least one of tissue contrast and instantaneous blood plasma contrast and, for any measured tissue contrast, subtracting said baseline tissue contrast and, for any measured blood plasma contrast, subtracting said baseline blood plasma contrast.

13. The method of claim 1 wherein said estimated hemodynamic parameters comprise contrast distribution volume (V_{D}), blood plasma space volume (Vp), and blood flow and extraction efficiency product (FE).

14. The method of claim 8 wherein, for a given image acquisition time, said measure related to an integral of tissue contrast enhancement with respect to time and said measure related to an integral of blood plasma contrast enhancement with respect to time comprises an area bounded by a contrast enhancement measured at said given image acquisition time.

15. The method of claim 1 wherein there are at least three time sequenced images.

16. The method of claim 1 wherein there are four or more time sequenced images.

17. The method of claim 8 wherein said obtaining a measure comprises iteratively solving the following system of linear equations:
$$[ \begin{matrix}{A}_{Q} ( {T}_{1} ) \\ {A}_{Q} ( {T}_{2} ) \\ ⋮ \\ ⋮ \\ ⋮ \\ {A}_{Q} ( {T}_{n} )\end{matrix} ] = [ \begin{matrix}{A}_{p} ( {T}_{1} ) & - Q ( {T}_{1} ) & {C}_{p} ( {T}_{1} ) \\ {A}_{p} ( {T}_{2} ) & - Q ( {T}_{2} ) & {C}_{p} ( {T}_{2} ) \\ & ⋮ & \\ & ⋮ & \\ & ⋮ & \\ {A}_{p} ( {T}_{n} ) & - Q ( {T}_{n} ) & {C}_{p} ( {T}_{n} )\end{matrix} ] [ \begin{matrix}{V}_{D} \\ {k}^{- 1} \\ {V}_{p} \cdot {k}^{- 1}\end{matrix} ]$$
where: A_{Q} is an area under a curve of contrast enhancement with respect to time for interstitial space
A_{P} is an area under a curve of contrast enhancement with respect to time for blood plasma space
Q is tissue contrast enhancement
Cₚ is blood plasma enhancement
Tₙ is a time when an image was acquired
n is at least three, and
k=Fe/Vₑ

18. The method of claim 17 wherein n is four or more.

19. The method of claim 18 wherein said time sequenced images are computer tomography images.
